(19) United States
(12) Patent Application Publication
(10) Pub. No.: US 2012/0196233 A1
(43) Pub. Date: Aug. 2, 2012

(54) RECOVERY OF DISSOLVED ORGANICS FROM LIGNOCELLULOSIC SOLUTIONS

(76) Inventors: Yonghao NI, Fredericton (CA); Pedram FATEHI, Fredericton (CA); Jing SHEN, Fredericton (CA)

(21) Appl. No.: 13/361,204

(22) Filed: Jan. 30, 2012

Related U.S. Application Data
(60) Provisional application No. 61/438,402, filed on Feb. 1, 2011.

(51) Int. Cl. C10L 1/12; C07C 51/47; C13K 13/00; C07G 1/00

(52) U.S. Cl. 431/2; 530/500; 562/608; 127/44

(57) ABSTRACT

Methods are provided for the recovery of dissolved organics, such as hemicelluloses, lignin, and acetic acid, from a lignocellulosic feedstock or process liquor, where the dissolved organics are recovered via an adsorbent. The adsorbent may include activated carbon, modified activated carbon, precipitated calcium carbonate, and lime and/or lime mud. The dissolved organics may be adsorbed from a pre-hydrolysis liquor of a pulping process such as the Kraft-based dissolving pulp production process. Other methods include a combined (or integrated) process of adsorption, ion exchange resin treatment, and membrane filtration for the treatment of a lignocellulosic liquor, such as a pre-hydrolysis liquor of kraft-based dissolving pulp production process, such that dissolved organics such as lignin, acetic acid, and hemicellulose-derived sugars may be extracted and optionally concentrated.

### CROSS-REFERENCE TO RELATED APPLICATION

This application claims priority to U.S. Provisional Application No. 61/438,402, titled “RECOVERY OF DISSOLVED ORGANICS IN PRE-HYDROLYSIS LIQUOR BY ADSORPTION” and filed on Feb. 1, 2011, the entire contents of which are incorporated herein by reference.

### BACKGROUND

This disclosure relates to the processing of lignocellulosic feedstock and process liquors for the extraction of lignocellulosic organics.

With the ever-increasing demand for energy, fuels, chemicals, and materials, fossil-based resources have been extensively exploited to meet the requirements of the development of various industries. However, there is a limit that fossil-based resources can be exploited, as they are exhaustible and non-renewable. Also, the continuing use of these resources contributes to global warming, which has received more and more attention globally. In this context, the utilization of renewable green natural resources for the production of energy, fuels, chemicals, and materials, widely referred to as “biorefinery”, is of strategic significance.

As a commercially available process, the kraft-based dissolving pulp production process utilizes a pre-hydrolysis step to remove hemicelluloses/others from lignocellulosic materials. This step is rather critical for the production of dissolving pulp (pulp with a very high cellulose content, >90%), mainly due to the fact that impurities, including hemicelluloses, can precipitate onto the cellulose micro-fibrils and cause operational and quality issues during the downstream processes associated with dissolving pulp [1,2]. The kraft pulping and bleaching steps are applied to remove lignin and obtain relatively pure cellulose.

For the pre-hydrolysis of lignocellulosic materials, other organics including lignin, acetic acid, and hemicelluloses/sugars/furfural can also be formed in the pre-hydrolysis liquor. The recovery and utilization of these valuable dissolved organics in pre-hydrolysis liquor is important for generating additional revenue for the mills. Hemicelluloses can be a valuable source of hexose and pentose sugars, and they can be further converted into value-added products such as ethanol, polymers and other chemicals. Another potentially useful product present in the pre-hydrolysis liquor is lignin. Lignin can be used as a solid fuel, or as raw material for other products, such as plastics.

Unfortunately, the relatively low concentration of dissolved lignocellulosic materials in the pre-hydrolysis liquor hinders their practical application in various downstream processes, and the conventional evaporation process is very costly.

### SUMMARY

Methods are provided for the recovery of dissolved organics, such as hemicelluloses, lignin, and acetic acid, from a lignocellulosic feedstock or process liquor, where the dissolved organics are recovered via an adsorbent. The adsorbent may include activated carbon, modified activated carbon, precipitated calcium carbonate, and lime and/or lime mud. The dissolved organics may be adsorbed from a pre-hydrolysis liquor of a pulping process such as the Kraft-based dissolving pulp production process. Other methods include a combined (or integrated) process of adsorption, ion exchange resin treatment, and membrane filtration for the treatment of a lignocellulosic liquor, such as a pre-hydrolysis liquor of kraft-based dissolving pulp production process, such that dissolved organics such as lignin, acetic acid, and hemicellulose-derived sugars may be extracted and optionally concentrated.

Accordingly, in a first embodiment, there is provided a method of treating a solution containing lignocellulosic material, the method comprising the steps of: providing a quantity of activated carbon; producing modified activated carbon by contacting the activated carbon with one of an oxidizing agent and a cationic polymer; mixing the modified activated carbon with the solution for a time duration suitable for adsorbing lignocellulosic material onto the modified activated carbon; and separating the modified activated carbon having the lignocellulosic material adsorbed thereon from the solution.

In another embodiment, there is provided a method of extracting lignocellulosic organic material from a lignocellulosic solution, the method comprising the steps of: providing a quantity of calcium carbonate; forming a mixture including the lignocellulosic solution and calcium carbonate; providing a quantity of a cationic polymer, the cationic polymer being selected for inducing flocculation of the lignocellulosic organic material; adding the cationic polymer to the mixture and mixing for a time duration suitable for adsorbing lignocellulosic organic material onto the calcium carbonate; and separating the calcium carbonate from the mixture, thereby obtaining calcium carbonate having the lignocellulosic organic material adsorbed thereon.

In another embodiment, there is provided a method of extracting lignocellulosic organic material from a lignocellulosic solution, the method comprising the steps of: providing a quantity of lime; forming a mixture including the lignocellulosic solution and the lime; providing a quantity of a cationic polymer, the cationic polymer being selected for inducing flocculation of the lignocellulosic organic material; adding the cationic polymer to the mixture and mixing for a time duration suitable for adsorbing lignocellulosic organic material onto the lime; and separating the lime from the mixture, thereby obtaining calcium carbonate having the lignocellulosic organic material adsorbed thereon.

In another embodiment, there is provided a method of treating a lignocellulosic solution, the lignocellulosic solution containing lignocellulosic organic material having a molecular weight between approximately 700 and 10,000, acetic acid, and hemicellulose sugars, the method comprising the steps of: providing a quantity of adsorbent configured to adsorb the lignocellulosic organic material; mixing adsorbent with the lignocellulosic solution for a time duration suitable for adsorbing a substantial portion of the lignocellulosic organic material onto the adsorbent; separating the adsorbent from the lignocellulosic solution; providing a quantity of an ion exchange resin, wherein the ion exchange resin is configured to adsorb acetic acid; contacting the ion exchange resin with the lignocellulosic solution and extracting acetic acid onto the ion exchange resin; separating the ion exchange resin from the lignocellulosic solution; providing a filter configured to capture hemicellulose sugars; and filtering the lignocellulosic solution with the filter, thereby concentrating the hemicellulose sugars; wherein the step of mixing the adsorbent with the lignocellulosic solution is performed prior to the steps of contacting the ion exchange resin with the lignocellulosic solution and filtering the lignocellulosic solution with the filter, such that the lignocellulosic organic material is removed prior to the removal of acetic acid and hemicellulose sugars.

A further understanding of the functional and advantageous aspects of the disclosure can be realized by reference to the following detailed description and drawings.

### BRIEF DESCRIPTION OF THE DRAWINGS

Embodiments will now be described, by way of example only, with reference to the drawings, in which:

FIG. 1 illustrates a process flow diagram in which activated carbon is employed as an adsorbent for extraction of lignocellulosic materials from pre-hydrolysis liquor.

FIG. 2 provides a process flow diagram in which calcium carbonate is employed as an adsorbent for the extraction of lignocellulosic materials from pre-hydrolysis liquor.

FIG. 3 is a flow diagram in which lime/lime mud is employed for the extraction of lignocellulosic materials from pre-hydrolysis liquor.

FIG. 4 is a flow chart illustrating a combined method of extracting lignin, acetic acid, and hemicellulose sugars from a lignocellulosic solution.

FIG. 5 plots adsorption isotherms of (a) hemicelluloses, (b) lignin, and (c) furfural on unmodified or modified activated carbon (1 g activated carbon to various amounts of pre-hydrolysis liquor at room temperature for 24 h).

FIG. 6 plots adsorption of (a) hemicelluloses, (b) lignin, and (c) furfural of pre-hydrolysis liquor (pH=7) on activated carbon versus time of adsorption. (1 g activated carbon was added to 120 mL of pre-hydrolysis liquor at room temperature).

FIG. 7 plots adsorption of (a) hemicelluloses, (b) lignin, and (c) furfural on activated carbon versus the dosage of PDADMAC or chitosan applied for the modification of activated carbon (pre-hydrolysis liquor 20 mL, 1 g of modified activated carbon, room temperature, 24 h).

FIG. 8 plots adsorption (a) hemicelluloses, (b) lignin, and (c) furfural on activated carbon versus the dosage of PDADMAC or chitosan applied for the modification of activated carbon (pre-hydrolysis liquor 20 mL, 1 g of modified activated carbon, room temperature, 24 h).

FIG. 9 plots the adsorption of (a) hemicelluloses, (b) lignin, and (c) furfural on PCCs versus the dosage of PDADMAC (based on pre-hydrolysis liquor) in the system.

FIG. 10 is a graph showing the effect of activated carbon treatment on the concentrations of dissolved organics in pre-hydrolysis liquor, plotting (a) amount of activated carbon relative to treated pre-hydrolysis liquor of 1/30 g/g and (b) amount of activated carbon relative to treated pre-hydrolysis liquor of 1/40 g/g.

FIG. 11 is a graph showing the effect of anion exchange resin treatment on the concentration of oligomeric sugars (resin treatment of activated carbon treated pre-hydrolysis liquor).

FIG. 12 is a graph showing the effect of anion exchange resin treatment on the concentration of monomeric sugars (resin treatment of activated carbon treated pre-hydrolysis liquor).

FIG. 13 is a graph showing the effect of anion exchange resin treatment on the acetic acid concentration (resin treatment of activated carbon treated pre-hydrolysis liquor).

FIG. 14 is a graph showing the effect of anion exchange resin treatment on lignin concentration (resin treatment of activated carbon treated pre-hydrolysis liquor).

FIG. 15 is a graph showing the nanofiltration membrane filterabilities of deionized water, untreated pre-hydrolysis liquor, activated carbon treated pre-hydrolysis liquor, and double-treated (activated carbon-treated and ion exchange resin-treated) pre-hydrolysis liquor.

FIG. 16 is a graph showing variations in oligomeric sugar concentrations of permeate and concentrate as a function of time.

FIG. 17 is a graph showing variations in monomeric sugar concentrations of permeate and concentrate as a function of time.

FIG. 18 is a graph showing variations in lignin concentrations of permeate and concentrate as a function of time.

FIG. 19 is a graph showing variations in acetic acid concentrations of permeate and concentrate as a function of time.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure. It should be understood that the order of the steps of the methods disclosed herein is immaterial so long as the methods remain operable. Moreover, two or more steps may be conducted simultaneously or in a different order than recited herein unless otherwise specified.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein, the terms “about” and “approximately”, when used in conjunction with ranges of dimensions of particles, compositions of mixtures or other physical properties or characteristics, are meant to cover slight variations that may exist in the upper and lower limits of the ranges of dimensions so as to not exclude embodiments where on average most of the dimensions are satisfied but where statistically dimensions may exist outside this region. It is not the intention to exclude embodiments such as these from the present disclosure.

Some embodiments of the present disclosure provide methods for the recovery of dissolved organics, such as hemicelluloses, lignin, and furfural, from a lignocellulosic feedstock or process liquor, where the dissolved organics are recovered via an adsorbent. The adsorbent may include activated carbon, modified activated carbon, magnesium hydroxide/magnesium oxide, precipitated calcium carbonate, lime, lime mud, bentonite and zeolite. The methods disclosed herein may be employed for the absorption of dissolved organics from a pre-hydrolysis liquor of a pulping process such as the Kraft-based dissolving pulp production process.

In one embodiment, the adsorbent is mixed with pre-hydrolysis liquor to adsorb lignocellulosic materials under various conditions, as will be further described below. Subsequently, desorption of lignocellulosic materials from the adsorbent may be achieved via a chemical treatment. In embodiments in which the adsorbed lignocellulosic materials are to be applied in downstream processes to produce value-added chemicals, the adsorbent may be reused, thus providing a potentially integrated process with the advantages of simplicity and recyclability. In another embodiment, where the recovered organics from the adsorption process are suitable as a fuel source, they can be directly burned so to achieve combustion, and the adsorbent may be retained as solid residue, and optionally reused for additional processing.

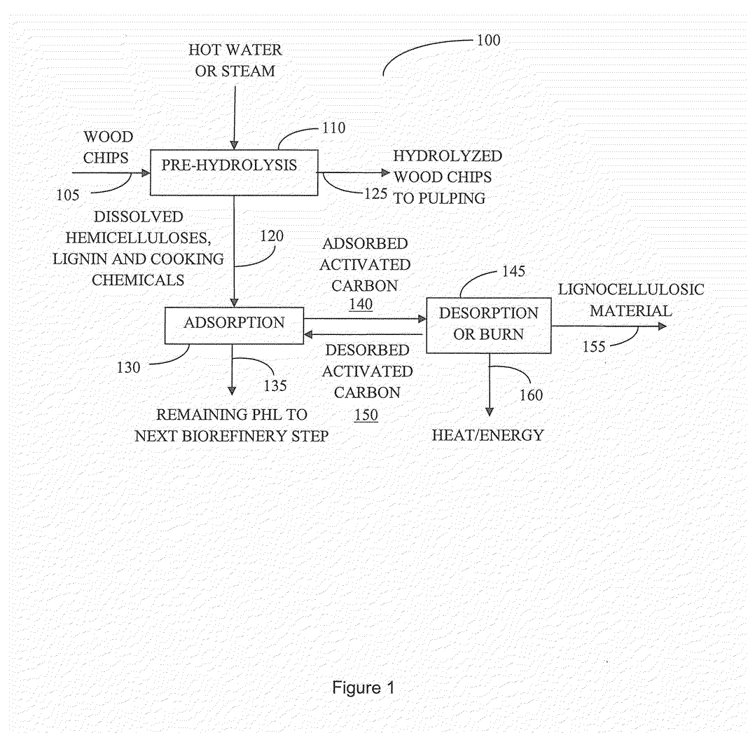

Referring now to FIG. 1, a process diagram is provided that illustrates an example method in which activated carbon is employed as an adsorbent for recovering a lignocellulosic organic material from a pre-hydrolysis liquor. In process 100, the activated carbon is mixed with a pre-hydrolysis liquor to adsorb the lignocellulosic organic material. To improve the adsorption, the activated carbon may be modified prior to the adsorption process, as further described below. Subsequently, the desorption of lignocellulosic organic materials from the activated carbon may be achieved in a chemical extraction step. Once the adsorbed organics are removed/processed, the activated carbon can be reused and recycled. Alternatively, the modified activated carbon can be burned, which produces a net heat in the system that may be employed as an energy source.

Example process 100 involves the pre-hydrolysis 110 of wood chips 105, producing hydrolyzed wood chips 125 with depleted hemicelluloses and lignin for improved performance in a subsequent pulping process (such as the Kraft process). The pre-hydrolysis liquor 120 obtained from pre-hydrolysis process 110 is processed in an adsorption process 130, in which lignocellulose organics such as hemicellulose, lignin and furfural are extracted by activated carbon. The depleted pre-hydrolysis liquor 135 is provided to a subsequent biorefinery step for further processing.

The adsorption process may be carried out by contacting a quantity of activated carbon with the pre-hydrolysis liquor, while mixing for a time duration that is sufficient to adsorb a desired portion of the dissolved lignocellulosic organics onto the activated carbon. In one example, approximately 1 to 100 g of activated carbon is employed per liter of pre-hydrolysis liquor. In another example, approximately 1 to 50 g of activated carbon is employed per liter of pre-hydrolysis liquor. The amount of activated carbon for a given application will generally depend on the concentration of the lignocellulosic organics.

As shown in the examples below, the time duration for mixing the activated carbon with the pre-hydrolysis liquor for obtaining a substantial degree of adsorption, relative to a long-time steady-state value, may be less than an hour. However, a suitable mixing time for obtaining a particular amount of adsorption may differ depending on the nature of the pre-hydrolysis liquor. It is to be understood that one skilled in the art, aided by the present disclosure, may determine a suitable time duration for mixing, and/or a suitable relative weight fraction of activated carbon.

Referring again to FIG. 1, the activated carbon 140, having adsorbed thereon lignocellulosic organics, is subsequently separated, for example, by filtration, and processed in step 145, and the desorbed activated carbon 150 is optionally recycled for further use. In one embodiment, the adsorbed lignocellulosic organics are desorbed from the activated carbon and recovered as product 155. Desorption of the lignocellulosic organics may be achieved by a chemical treatment such as acid hydrolysis or solvent extraction, such as ethyl acetate.

Alternatively, the activated carbon, having the lignocellulosic organic adsorbed thereto, may be burned, for example, in a gasifier. In one embodiment, the energy obtained from the combustion step can be employed as power source for one or more process steps, producing heat or another form of energy as product 160. The residual activated carbon obtained after the thermal process may be recycled (e.g. directly recycled) for subsequent extraction of additional lignocellulosic organics, provided that the oxygen supply during combustion is less than the amount required for complete combustion.

Prior to performing the adsorption step 130 in the process outlined above, the pre-hydrolysis liquor may be acidified to induce lignin precipitation. The precipitated lignin may be collected in a pre-extraction step, for example, by filtration. In one embodiment, the acidification is performed at a pH of approximately 2 by the addition of sulphuric acid.

In one example implementation, the pre-hydrolysis liquor may be treated or pre-treated to obtain an appropriate pH for the subsequent adsorption step employing the activated carbon. One example pH range for performing the adsorption step is 5-10. In another example, a pH of 6-8 may yield improved results. For example, in circumstances in which the pre-hydrolysis liquor is acidic (for example, when the preceding acidification step is performed), the pre-hydrolysis liquor may be neutralized, for example, by the addition of a basic substance such as calcium oxide.

The activated carbon may be modified by using an oxidant or a cationic polymer, to provide improved adsorption and recovery of dissolved lignocellulosic organics. For example, in one embodiment, the activated carbon may be oxidized by an oxidizing agent. Suitable oxidizing agents include, but are not limited to, hydrogen peroxide, sulphuric acid, peroxy acid, and nitric acid. The oxidation of the modified carbon may be performed by mixing the activated carbon with a solution containing the oxidizing agent for a time duration and a temperature that is suitable for producing the desired degree of modification. For example, as further described in the Examples below, the activated carbon may be mixed in a solution of H₂O₂ having a concentration of approximately 0.1 to 10 g/l, for a time duration of approximately 5 to 90 min, at a temperature of approximately 25 to 70 degrees centigrade, and at a weight percentage of activated carbon to solution of 1 to 20% The oxidized activated carbon may be subsequently washed and dried before being employed in the adsorption step.

In another embodiment, the activated carbon may be modified by a cationic polymer. The cationic polymers may be flocculent/coagulants. Non-limiting examples of cationic polymers include polydiallyldimethylammonium chloride (PDADMAC), chitosan, poly acrylamide, poly aluminum chloride, and other amine-based cationic polymers, and their combinations. The relative amount of cationic polymer employed in the process may vary depending on the desired extraction efficiency. In one non-limiting example implementation, it may be approximately 2-2.5 milligrams per gram of activated carbon. The activated carbon, after having been contacted with the cationic polymer, may be subsequently washed and dried before being employed in the adsorption step.

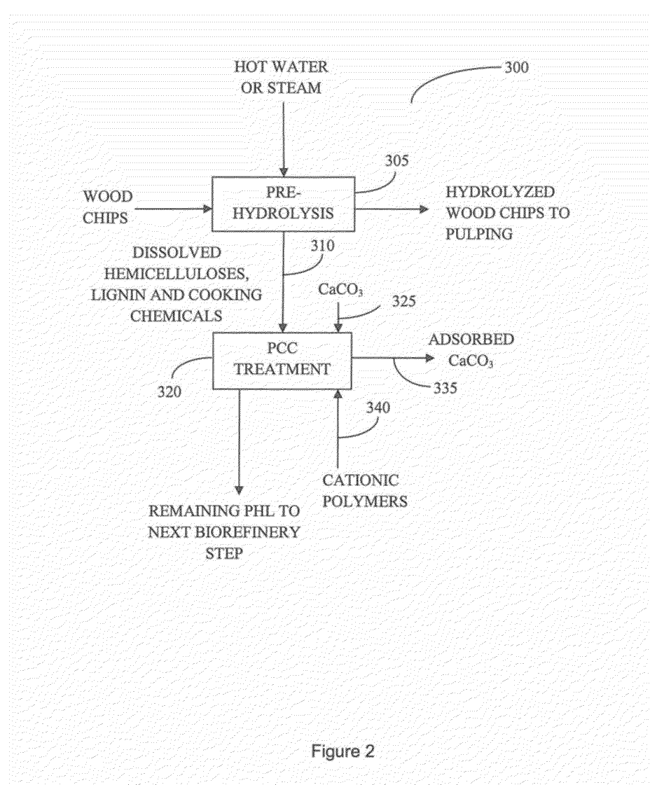

In another embodiment, precipitated calcium carbonate (PCC) may be employed as an adsorbent. Referring to FIG. 2, a process diagram is provided in illustrating the use of calcium carbonate for the recovery of lignocellulosic organics from pre-hydrolysis liquor. As in the preceding embodiment, in process 300, a pre-hydrolysis step 305 is performed and pre-hydrolysis liquor including dissolved hemicelluloses, lignin and cooking chemicals 310 is obtained.

The pre-hydrolysis liquor is subsequently processed in an adsorption treatment 320 in which the pre-hydrolysis liquor is contacted with precipitated calcium carbonate 325 (while mixing) for the adsorption of lignocellulosic organics. The adsorbed calcium carbonate 335, having adsorbed lignocellulosic organics, may be subsequently processed to remove and recover the adsorbed organics, for example, by a filtration step. Suitable example processes for desorbing the lignocellulosic organics from the calcium carbonate include pH adjustment and solvent extraction.

The precipitated calcium carbonate may include nano-size precipitated calcium carbonate and/or porous precipitated calcium carbonate. The amount of precipitated calcium carbonate for a given application may vary according to the concentration of lignocellulosic organics in solution. In one example, 0.5 to 100 g of precipitated calcium carbonate is employed per liter of pre-hydrolysis liquor. In another example, 1 to 8 g of precipitated calcium carbonate is employed per liter of pre-hydrolysis liquor.

To further improve adsorption, cationic polymers 340 can also be applied in the calcium carbonate adsorption process step 320. The cationic polymers interact with the lignocellulosic materials of pre-hydrolysis liquor, forming flocculants that can readily be adsorbed on the calcium carbonate particles. The concentration of cationic polymers may vary according to the degree of flocculation required. The cationic polymers may be provided with an amount in the range of 0.1 to 10 g per liter of pre-hydrolysis liquor. In another example, the cationic polymers may be provided with an amount in the range of 1 to 10 g per liter of pre-hydrolysis liquor. Generally speaking, the cationic polymers listed above in the activated carbon process example may also be employed in the present calcium carbonate embodiment. In an alternative embodiment, the cationic polymers may be mixed with the pre-hydrolysis liquor prior to the calcium carbonate adsorption step 320.

The time duration for mixing the precipitated calcium carbonate with the pre-hydrolysis liquor (and optionally with the cationic polymers) for obtaining a substantial degree of adsorption, relative to a long-time steady-state value, may be less than an hour. However, a suitable mixing time for obtaining a particular amount of adsorption may differ depending on the nature of the pre-hydrolysis liquor. It is to be understood that one skilled in the art, aided by the present disclosure, may determine a suitable time duration for mixing, and/or a suitable relative weight fraction of precipitated calcium carbonate (and optionally cationic polymer), through routine experimentation.

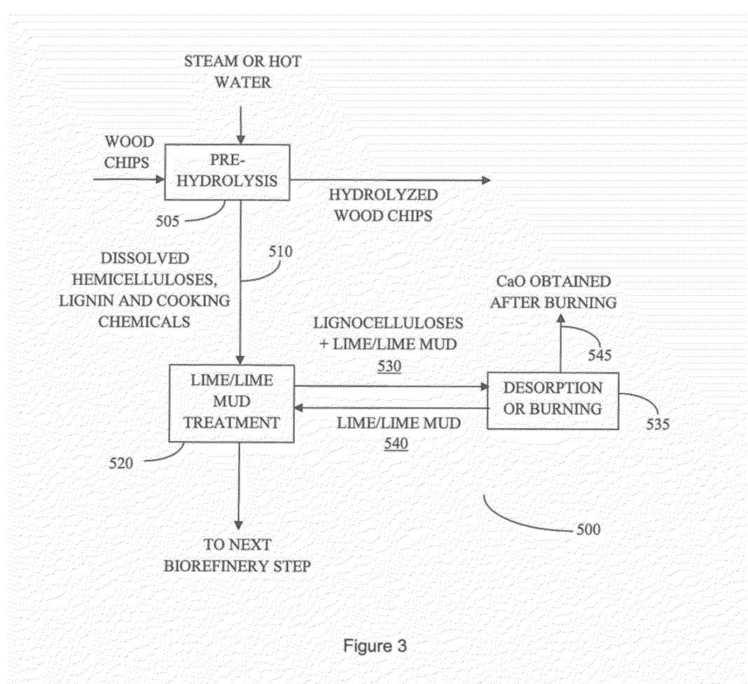

In another embodiment, illustrated in example process 500 shown in FIG. 3, Ca(OH)₂ may be employed as an adsorbent. A pre-hydrolysis step 505 is performed and pre-hydrolysis liquor comprising dissolved hemicelluloses, lignin and cooking chemicals 510 is obtained. In step 520, the pre-hydrolysis liquor is treated with lime and/or lime mud. The presence of insoluble lime/lime mud facilitates the adsorption of lignocellulosic materials present in the pre-hydrolysis liquor. In a manner similar to that described in the previous embodiment (involving precipitated calcium carbonate), cationic polymers may be added so that the complexes (flocculants) are formed as a result of the interaction between the cationic polymers and lignocellulosic materials in the pre-hydrolysis liquor. In an alternative embodiment, the cationic polymers may be mixed with the pre-hydrolysis liquor prior to step 520. The Ca(OH)₂ may be produced in the lime kiln of a pulp processing mill.

The lime and/or lime mud with adsorbed lignocellulosic organics 530 is separated, for example, via filtration, and provided to recovery unit 535, where the lignocellulosic organics may be recovered by a solvent extraction unit. The treated lime can be alternatively burned in the lime kiln of the kraft mill so that calcium oxide 545 is regenerated and the burning of lignocellulosic materials provides a net heat in the system. In this case, the produced heat decreases the heat requirement of the lime kiln in the kraft process. The desorbed lime and/or lime mud 540 is optionally returned to treatment process 520 for further extraction.

It is to be understood that a suitable relative weight fraction of lime and/or lime mud will depend on the nature of the pre-hydrolysis liquid (such as the pH, temperature, and/or concentration of the dissolved lignocellulosic organic materials). In one example, the weight percent of lime and/or lime mud relative to the pre-hydrolysis liquor may be in the range of 0.5 to 100 g per liter of pre-hydrolysis liquor, or, in another example, in the range of 0.5 to 50 g per liter of pre-hydrolysis liquor. The cationic polymers may be provided with an amount in the range of 0.1 to 10 g per liter of pre-hydrolysis liquor. In another example, the cationic polymers may be provided with an amount in the range of 1 to 10 g per liter of pre-hydrolysis liquor.

Although the preceding embodiments and the examples below pertain to the treatment of pre-hydrolysis liquor, it is to be understood that the methods described herein may be applied for the separation and optional recovery of lignocellulosic organic materials from other lignocellulosic solutions, feedstocks or process liquors. For example, the lignocellulosic solution may be a waste stream from a lignocellulosic process, including the spent liquor/filtrate of organosolv pulping, sulfite pulping, and neutral sulfite semichemical process (NSSC).

In other embodiments of the present disclosure, methods are provided for the extraction of dissolved organics in a pre-hydrolysis liquor according to a three-step process. As will be described below, the present methods are well suited for the extraction, and also the recovery, of lignin, acetic acid, and hemicelluloses from a pre-hydrolysis liquor. In particular, the methods disclosed below may be configured for the recovery, concentration, and purification of dissolved organics, including sugars, acetic acid, and lignin, in the pre-hydrolysis liquor of kraft-based dissolving pulp production process. Although the example embodiments provided below relate to the extraction of dissolved organics from a pre-hydrolysis liquid, it is to be understood that the methods are not intended to be restricted to pre-hydrolysis liquor, and may be adapted, based on the present disclosure, to be applicable to other hydrolysis liquor/pre-extract, or other waste streams from processes related to lignocellulosic material treatment and production.

As will be further described below, it has been found that by using the combined process of adsorption, ion exchange resin treatment, and membrane filtration (or separation or concentration), dissolved organics within a lignocellulosic solution can be effectively recovered and concentrated, which facilitates their downstream processing and utilization to produce various value-added products. In the proceeding disclosure, it is to be understood that membrane filtration may be employed for the extraction, optional separation, and optional concentration of a substance.

While not wishing to be bound by any particular theory, the combined process of adsorption, ion exchange resin treatment, and membrane filtration for the recovery/purification of dissolved organics may involve various individual or interrelated mechanisms, such as mechanical attachment, intermolecular bonding, covalent bonding, competitive surface encapsulation/anchoring, electrostatic attraction, hydrogen bonding, chemical precipitation and deposition, and colloidal interactions.

In one embodiment, a method for the recovery of dissolved lignocellulosic organics from a lignocellulosic solution is provided in which an adsorption step is initially performed for the adsorption and separation of lignin, followed by at least one of ion exchange resin treatment and membrane filtration/separation/concentration for the recovery of acetic acid and hemicellulose sugars (monomeric/oligomeric sugars), respectively.

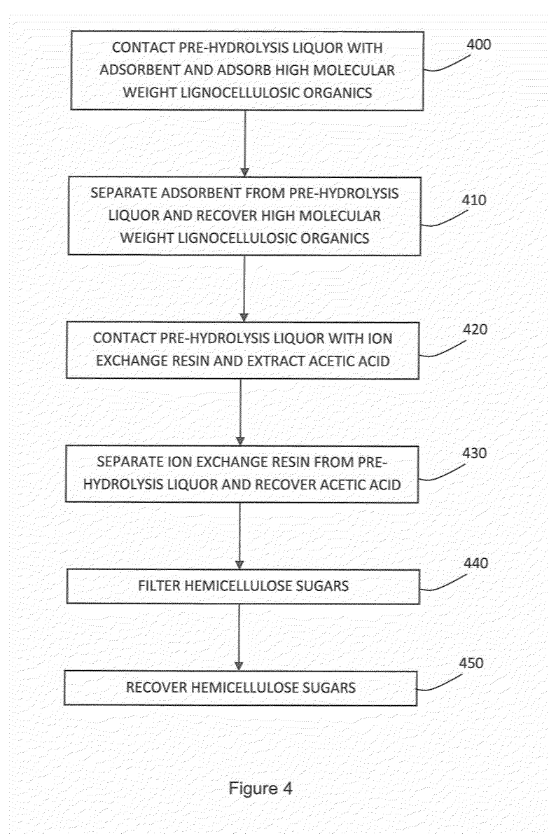

Referring to FIG. 4, an example method is illustrated in which a pre-hydrolysis liquor containing high molecular weight lignocellulosic organics (such as lignin), acetic acid and hemicellulose sugars is first contacted with (i.e. mixed with) an adsorbent in step 400, where the adsorbent is configured for the adsorption of high molecular weight lignocellulosic organics such as lignin. The adsorbent is contacted with the pre-hydrolysis liquor for a time duration sufficient for adsorption of a substantial fraction of the high molecular weight lignocellulosic organics. In one example, the adsorbent is configured for the capture of dissolved lignocellulosic organics having a molecular weight within the range of approximately 700 to 10,000. The adsorbent may be provided as combinations of several adsorbents, or combinations of adsorbents and co-adsorbents. In one example embodiment, the adsorbent may be provided in a quantity of approximately 1 to 100 g per liter of pre-hydrolysis liquor.

Suitable adsorbents include, but are not limited to, activated carbon, modified activated carbon, calcium carbonate, bentonite, talc, kaolin clay, precipitated calcium sulphate, lime, lime mud, and any combination of the above, optionally with the inclusion of a cationic polymer for inducing flocculation (as also described above). Co-adsorbents can be any form of substances that are used to improve the adsorption of dissolved organics, these substances include but not limited to polyamide epichlorohydrin, polyethyleneimine, starch, gum, polyacrylamide, polyvinylamine, and polyisocyanate, and polydiallyldimethylammonium chloride, alum, and polyaluminium chloride. The adsorbents may be provided in a form that can be easily regenerated and/or reactivated, or the organics-modified adsorbents can be used as functional additives and composite materials in various industries, such as papermaking and plastic industries.

In step 410, the adsorbent is subsequently removed and the adsorbed high molecular weight lignocellulosic organic is optionally desorbed and optionally concentrated. As discussed below, the removal of the high molecular weight lignocellulosic organics improves the performance/operation of subsequent processes, for example, processing the pre-hydrolysis liquor with an ion exchange resin, and filtering the pre-hydrolysis liquor with a membrane (e.g. nanofiltration membrane and reverse osmosis membrane).

After having extracted the lignin in steps 400 and 410, the processed pre-hydrolysis liquor is further processed for the extraction of acetic acid and hemicellulose sugars. As shown in FIG. 4, the processed pre-hydrolysis liquor may be contacted and mixed with an ion exchange resin in step 420 for the removal of acetic acid. The ion exchange resin may be subsequently treated to release and optionally concentrate the acetic acid. The suitable ion exchange resins can be any form of ion exchange resins capable of removing acetic acid, from a lignocellulosic solution. The ion exchange resins may have tertiary, quaternary amino functional groups, or other groups that can be used to interact with acetic acid, and can be regenerated after use. Two or more ion exchange resins can be mixed before use. In one example embodiment, the quantity of ion exchange resin provided is approximately 0.1 to 20 g per liter of pre-hydrolysis liquor.

In step 430, the acetic acid attached to ion exchange resin may be recovered. Example recovery processes include direct distillation, reactive distillation, extraction distillation, and elution.

In step 440, the processed pre-hydrolysis liquor is filtered for the capture of hemicellulose sugars. The filter may be subsequently treated to remove and optionally concentrate the retained hemicellulose sugars (monomeric and/or oligomeric). The filter can be any form of membrane with varied pore sizes or molecular weight cut-offs (MWCOs) capable of concentrating/separating the dissolved hemicellulose sugars. Examples of membranes are ultrafiltration membrane, nanofiltration membranes, and reverse osmosis membranes.

As noted above, the membrane filtration step of the combined process may be conducted after the substantial removal of high-molecular-weight lignin or lignin-like substances through adsorption, and this step results in concentrated dissolved organics with concentrations ranging from 10%-50%.

The dissolved organics can also be separated and purified in the membrane filtration step, and the separation/purification efficiency is dependent upon several factors, including pore sizes of membranes, and molecule size distributions of dissolved organics. Those skilled in the art may select an appropriate filter based on the present disclosure and additional routine experimentation. Different types of membranes can also be used in combination to serve for varied purposes. The recovered sugars may be extracted from the filter in step 450, optionally concentrated, and optionally converted into various value-added products, such as furfural, ethanol, and xylitol.

Unlike previous implementations for the extraction of lignocellulosic materials from a lignocellulosic solution, the present combined process provides a technically and economically feasible process whereby the initial adsorption step provides the dual benefit of recovering high molecular weight dissolved lignocellulosic organics (such as lignin), and also pre-processed (i.e. cleaned up) feed stock so that the subsequent ion exchange resin treatment and membrane filtration steps are possible due to the substantial removal of high molecular weight dissolved organics. The initial adsorption step thus improves the performance and/or time duration of the ion exchange resin treatment step and the membrane filtration step.

Accordingly, it is to be understood that while the adsorption steps (steps 400 and 410) are to be performed first for the removal of dissolved high molecular weight lignocellulosic organics such as lignin, the subsequent steps of ion exchange resin treatment (steps 420 and 430) and filtering (steps 440 and 450) may be reversed in order, such that the filtering steps are performed prior to the ion exchange resin treatment steps. However, it may be beneficial to order the steps as shown in FIG. 4, since the ion exchange resin treatment may eliminate other substances such that the filterability of the pre-hydrolysis liquor during the filtration step is improved.

As noted above, the steps of adsorption, ion exchange resin treatment, and membrane filtration of the combined process can be implemented in various sequences. For example, adsorption can be the first step, followed by sequential steps of either membrane filtration and ion exchange resin treatment, or ion exchange resin treatment and membrane filtration. Furthermore, in addition to the steps of adsorption, ion exchange resin treatment, and membrane filtration, other steps such as distillation and extraction can also be included and/or integrated in the combined process to recover the lignocellulosic organics.

In one example implementation, the adsorption step of the combined process may be conducted as a batch process. In another example implementation, a continuous processes (e.g., fixed-bed or expanded bed) may be employed. The adsorption step can be conducted immediately after the discharge of pre-hydrolysis liquor from the digester, or combined/integrated with the pre-hydrolysis of lignocellulosic materials. Two or more adsorbents and co-adsorbents can be mixed before use.

Similarly, in one example implementation, the ion exchange resin treatment step of the combined process may be conducted a batch process, while in another example implementation, continuous processes (e.g., fixed-bed or expanded bed) may be employed.

In an additional embodiment of the present disclosure, concentrated/purified sugars obtained from the combined process can be utilized to produce various value-added products via chemical, physical, and/or enzymatic-biological routes. These products include furfural, xylitol, ethanol, functional papermaking additives, biomaterials, etc. Acetic acid recovered from the combined process can be directly used in various industries. Lignin can be utilized as a fuel during the regeneration/reactivation processes of adsorbents, or used to produce various platform chemicals.

The aforementioned combined processing embodiments may be employed for a wide range of lignocellulosic liquids containing heavy molecular weight lignocellulosic organics, acetic acid, and hemicellulose sugars. In one example embodiment, the lignocellulosic solution is a pre-hydrolysis liquor having the following composition: a solids content of approximately 4 to 12% by weight (more preferably approximately 5-9%); a pH of approximately 3-5 (more preferably approximately 3.5-4.5); and a chemical composition such that approximately 15-25% by weight of the dissolved organics are acetic acid, 25-45% by weight of the dissolved organics are sugars such as xylan/xylose and the majority of hemicelluloses are still in their polymeric/oligomeric form.

The following examples are presented to enable those skilled in the art to understand and to practice embodiments of the present disclosure. They should not be considered as a limitation on the scope of the present embodiments, but merely as being illustrative and representative thereof.

### EXAMPLES

### Example 1

### Adsorption of Lignocellulosic Materials of Pre-Hydrolysis Liquor on Activated Carbon

### Part (A): Adsorption on Oxidized Activated Carbon

### Pre-Hydrolysis Liquor, Unmodified and Modified Activated Carbon

Pre-hydrolysis liquor was collected from a commercial plant that produces dissolving pulp based on the Kraft process, which operates in Eastern Canada, and uses a mixture of maple, poplar, and birch as raw materials. The original pH of pre-hydrolysis liquor was 4.5. The pre-hydrolysis liquor was first acidified to a pH of 2 using sulfuric acid to induce the lignin precipitation [4]. After the acidification and filtration, the pre-hydrolysis liquor was neutralized to a pH of 7 by using calcium oxide.

The modification of activated carbon was carried out by using H₂O₂ under the conditions of 30° C., 20% (wt.) H₂O₂ concentration for 4 h. The modification of activated carbon was also carried out by using H₂SO₄ under the conditions of 40° C., 6% (wt.) concentration and 6 h. The reactions were conducted, in both cases, by adding activated carbon in an amount equal to approximately 4% by weight, in Erlenmeyer flasks. Afterwards, the modified activated carbon was washed with water, and dried in an oven at 105° C. for 4 h.

### Characterization

The concentration of sugars in the original pre-hydrolysis liquor and filtrates was determined using an ion chromatography unit equipped with CarboPac™ PA1 column (Dionex-300, Dionex Corporation, Canada) and a pulsed amperometric detector (PAD). The detailed procedure is given in the literature [5]. To convert oligosaccharide of pre-hydrolysis liquors to monosaccharide, an additional acid hydrolysis of the sample was carried out under the conditions of 4% sulfuric acid at 121° C. in an oil bath (Neslab Instruments, Inc., Portsmouth, N.H., USA) [6]. The PAD settings were E1=0.1 V, E2=0.6 V and E3=−0.8 V. Deionized water was used as the eluent with a flow rate of 1 mL/min, 0.2 N NaOH was used as the regeneration agent at a 1 mL/min flow rate and 0.5 N NaOH as the supporting electrolyte at a 1 mL/min flow rate. The samples were filtered and diluted prior to analysis.

The lignin content of the original pre-hydrolysis liquor and processed samples was measured based on the UV/Vis spectrometric method at a wavelength of 205 nm (TAPPI UM 250). A Varian 300 ¹H-NMR spectrometer was employed for determining the concentrations of furfural [7] and acetic acid [8,9]. Calibration curves were established for both furfural and acetic acid. The solvent suppression method was used with D₂O to water ratio of 1:4.

### Adsorption of Lignocellulosic Materials on Activated Carbon

The pre-hydrolysis liquor contained 2.03 g/l furfural, 8.24 g/l acetic acid, 11.78 g/l lignin and 23.11 g/l hemicelluloses at pH 7. The H₂O₂ or H₂SO₄ modification changed the available surface area and carboxylic group content of activated carbon. To investigate the adsorption isotherms, various amounts of the pre-hydrolysis liquor having a pH of 7 was added to 1 g of the activated carbon in 125 mL Erlenmeyer flasks and shaken at 150 rpm and room temperature for 24 h.

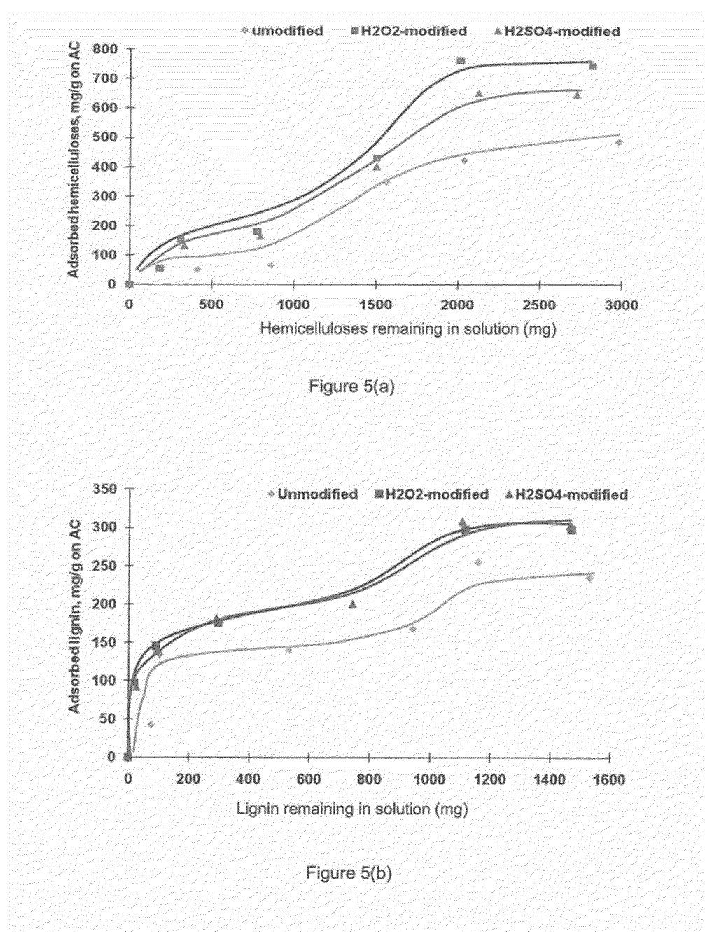

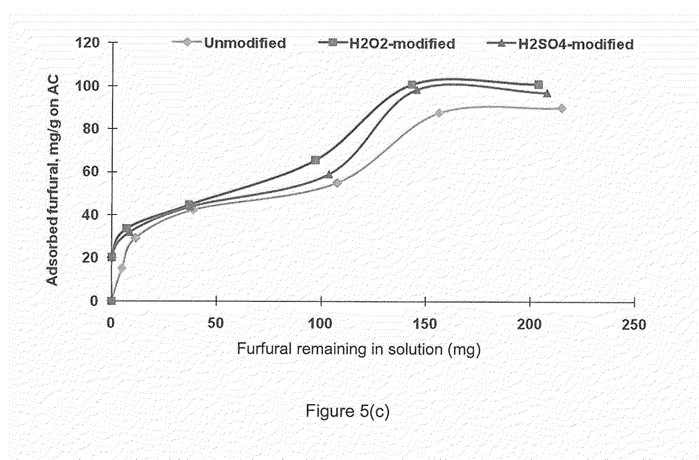

FIG. 5 shows the amount of dissolved materials adsorbed on activated carbon versus the amount remaining in the pre-hydrolysis liquor filtrates. It is evident that, the adsorption of hemicelluloses, lignin, and furfural, shown in (a), (b) and (c), respectively, reached to two levels of plateau. This behaviour may be due to the multilayer adsorption of lignocellulosic materials on the surface of activated carbon at different dosages of pre-hydrolysis liquor or to the diffusion of lignocellulosic materials into the pores of activated carbon [10].

The second plateau level for hemicelluloses, lignin, and furfural adsorption were 480.9 mg/g, 318.5 mg/g and 124.5 mg/g on the unmodified activated carbon. Interestingly, by oxidizing the activated carbon using H₂O₂ or H₂SO₄, the adsorption capacity of hemicelluloses increased to 761.27 mg/g or 600 mg/g, respectively. In the same vein, the adsorption of lignin increased to 300 mg/g (FIG. 5). However, the modification seems to affect the adsorption capacity of furfural to a less extent.

The results in FIG. 5 show that, by oxidation with H₂O₂ or H₂SO₄, the adsorption capacity of hemicelluloses, lignin, and furfural increased by 55%-75%, 50%, and 25%, respectively.

To investigate the adsorption kinetics, 120 mL of pre-hydrolysis liquor having a pH of 7 was added to 1 g of selected activated carbon, and shaken at 150 rpm and room temperature for various time intervals. The samples were filtered, and the filtrates were collected for analysis.

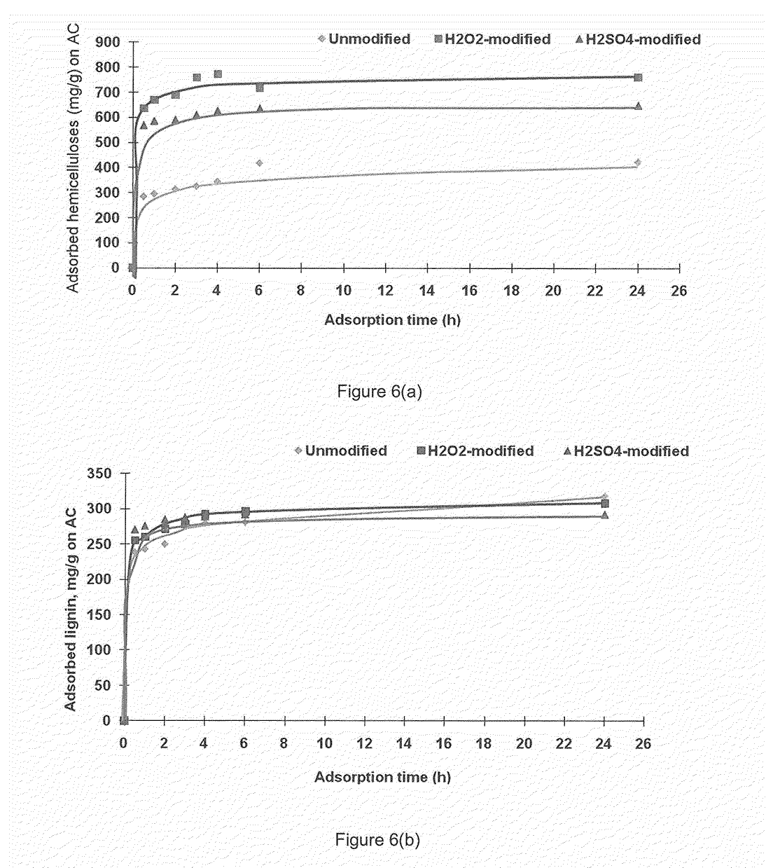

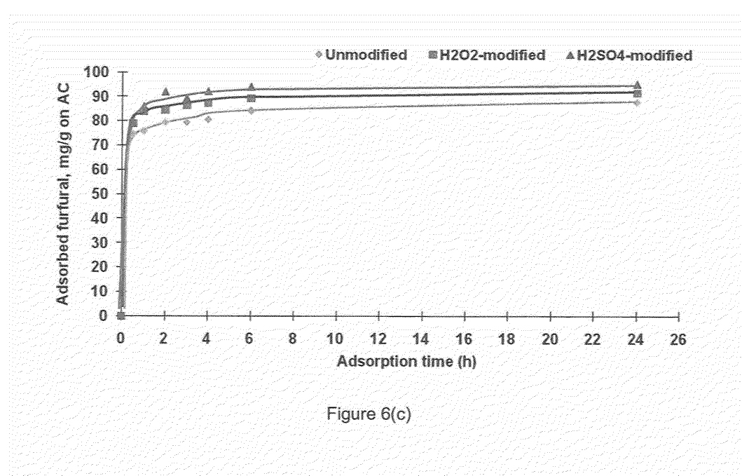

FIGS. 6(a), (b) and (c) show the adsorption of hemicelluloses, lignin, and furfural, respectively, versus the time of adsorption. It is evident that the adsorption reached the equilibrium in approximately 2 h. Furthermore, there are two distinct regimes for the adsorption of the materials on the activated carbon. In the first regime, i.e., <2 h, the adsorption reached 80-90% of its maximum capacity. In the second regime, i.e., >2 h, the adsorption amount gradually increased with a lower rate to the maximum. Without intending to be limited by theory, it may be assumed that the adsorption of lignocellulosic materials on the surface and large pores of activated carbon would occur in the first regime. In this step, most of the adsorption sites on the activated carbon were covered by the lignocellulosic materials. In the second regime, however, the adsorption increased via diffusion of the lignocellulosic materials into the smaller pores of activated carbon or the pores that were partly covered by the lignocellulosic materials in the first regime [10].

### Part (B): Adsorption on Polymer-Modified Activated Carbons

### Materials

The above-mentioned pre-hydrolysis liquor (pH 7) was used in this experiment. The polydiallyldimethylammonium chloride, PDADMAC (400,000 to 500,000 Mw), Potassium Polyvinyl Sulfate Titration Solution (PVSK), chitosan (70,000 to 180,000 Mw) were obtained from Aldrich Co.

### Modification of Activated Carbon by Polymers

To modify the activated carbon with polymers, various dosages of PDADMAC or chitosan solutions were added to 125 mL Erlenmeyer flasks that contained 2 g of activated carbon and deionized and distilled water, and the total volume was adjusted to 50 mL. The suspension were shaken at 150 rpm and 40° C. for 4 h. Afterwards, they were filtered and the filtrates were collected for further analysis, while the modified activated carbon were washed thoroughly with 500 mL of deionized and distilled water, dried in an oven at 105° C. for 4 h, and kept for adsorption of lignocellulosic materials dissolved in pre-hydrolysis liquor.

### Characterization

The characteristics of pre-hydrolysis liquor, hemicelluloses, lignin, and furfural content were determined as described in Part (A) above. To measure the adsorption of PDADMAC or chitosan on activated carbon, the concentration of PDADMAC or chitosan was measured before and after treating with activated carbon using a particle charge detector (Mütek PCD 03) and PVSK solution (1 mN). The difference in the concentration implies the amount of PDADMAC or chitosan adsorbed on activated carbon. This method has been widely applied in the literature to measure the adsorption of polymers on various surfaces [11].

### Adsorption of Lignocellulosic Materials on Polymer-Modified Activated Carbon

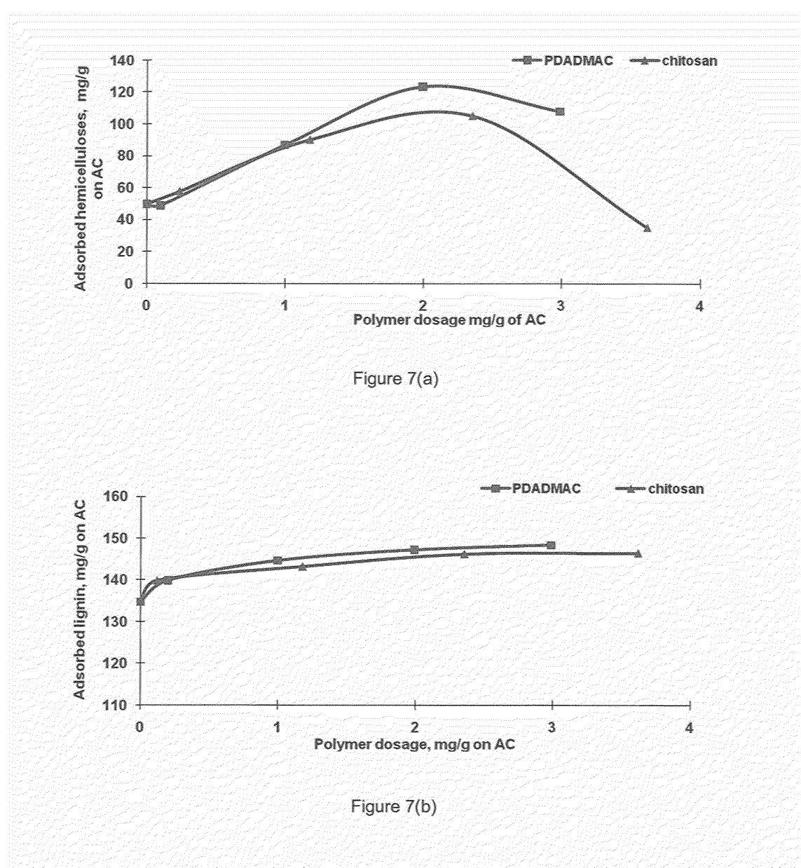

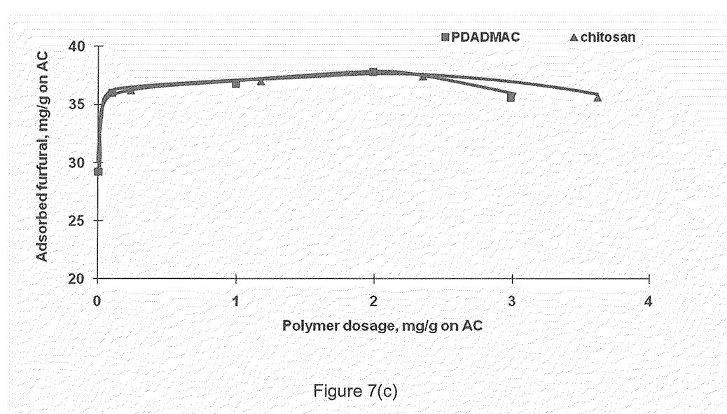

In one set of experiments, 20 mL of the pre-hydrolysis liquor having a pH of 7 was added to 1 g unmodified or modified activated carbon in 125 mL Erlenmeyer flasks and shaken at 150 rpm and room temperature for 24 h. The results of lignocellulosic materials adsorbed on activated carbon against the amount of PDADMAC or chitosan applied for modifying activated carbon are presented in FIG. 7. It is evident from FIG. 7(a) that by increasing the dosage of PDADMAC or chitosan up to 2 mg/g or 2.3 mg/g, the adsorption of hemicelluloses on activated carbon increased up to 123 mg/g or 100 mg/g, respectively. A further increase in the PDADMAC or chitosan dosage impaired the hemicelluloses adsorption. In the case of lignin and furfural adsorptions, shown in FIGS. 7(b) and 7(c), respectively, by adding 0.2 mg/g of PDADMAC or chitosan, their adsorptions increased to 140 and 36 mg/g, respectively, but further increasing the PDADMAC or chitosan dosage did not affect their adsorption.

Due to the maximum adsorption of lignocellulosic materials via adding 2 and 2.3 mg/g of PDADMAC or chitosan, respectively, these samples were selected as candidates for further analysis. To investigate the adsorption isotherms of lignocellulosic materials on various modified activated carbon, different dosages of the pre-hydrolysis liquor with the pH of 7 was added to 1 g of the selected modified activated carbon and shaken at 150 rpm and room temperature for 24 h.

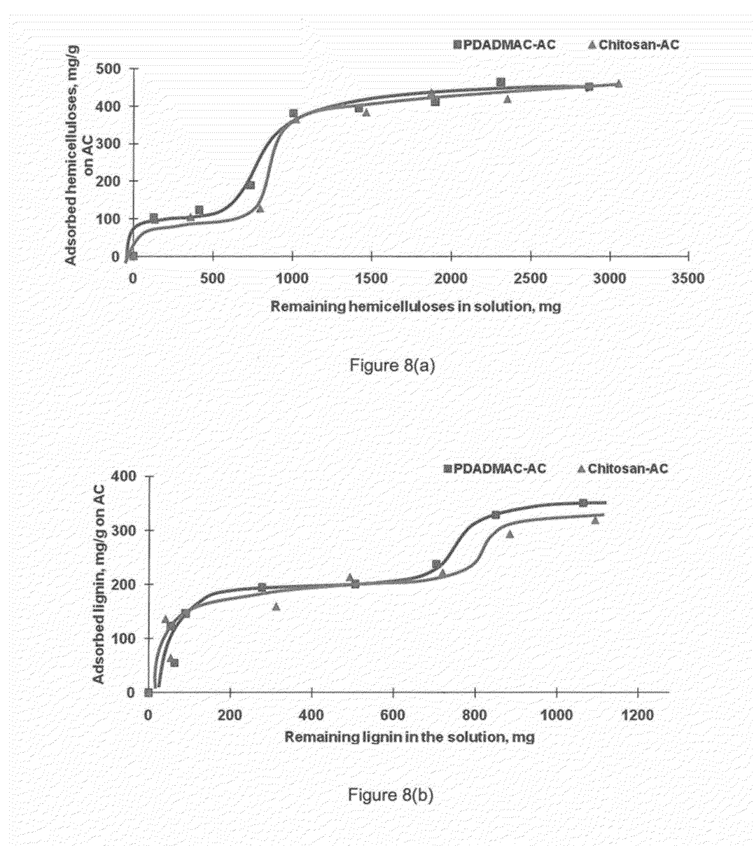

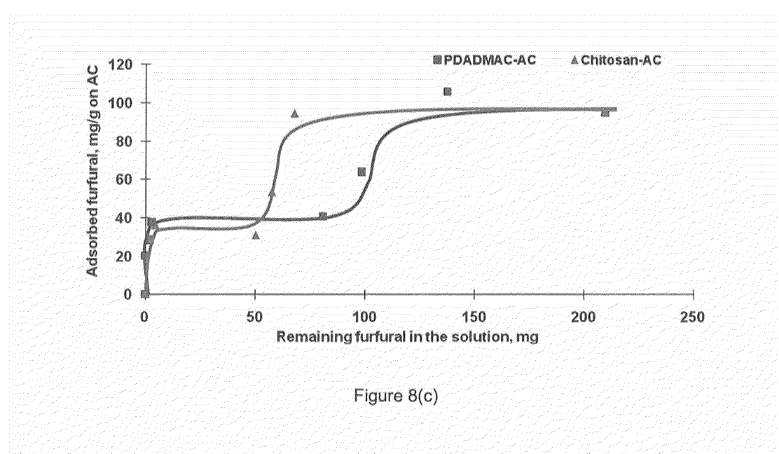

The adsorption isotherms of lignocellulosic materials on polymer-modified activated carbon are presented in FIG. 8. Generally, the adsorption of hemicelluloses, furfural and lignin, shown in FIGS. 8(a), (b) and (c), respectively, reached to two levels of plateau (similar to the adsorption of lignocellulosic materials on oxidized activated carbon). This behaviour may be due to the multi-layer adsorption of lignocellulosic materials on the surface of activated carbon or to their diffusion into the pores of activated carbon [10]. Evidently, PDADMAC- or chitosan-modified activated carbon had similar maximum adsorption level (FIG. 8).

### Example 2

### Adsorption of Lignocellulosic Materials of Pre-Hydrolysis Liquor on Calcium Carbonate

### Adsorption of Lignocellulosic Materials of Pre-Hydrolysis Liquor on Precipitated Calcium Carbonate

In the present example, the pre-hydrolysis liquor and PDADMAC used in Example 1 were employed and a precipitated calcium carbonate (PCC) sample was provided as an adsorbent [12]. Two types of precipitated calcium carbonate: 1) nano-size precipitated calcium carbonate (PCC1) and 2) porous precipitated calcium carbonate (PCC2) were used. In this set of experiments, 1 g of precipitated calcium carbonate was mixed with 50 g pre-hydrolysis liquor for 90 min at room temperature and 120 rpm. Subsequently, various dosages of PDADMAC were added to the precipitated calcium carbonate/pre-hydrolysis liquor system and mixed for additional 90 min. Subsequently, the suspensions were filtered according to the procedure described above and the remaining amount of lignocellulosic materials in the pre-hydrolysis liquor was determined.

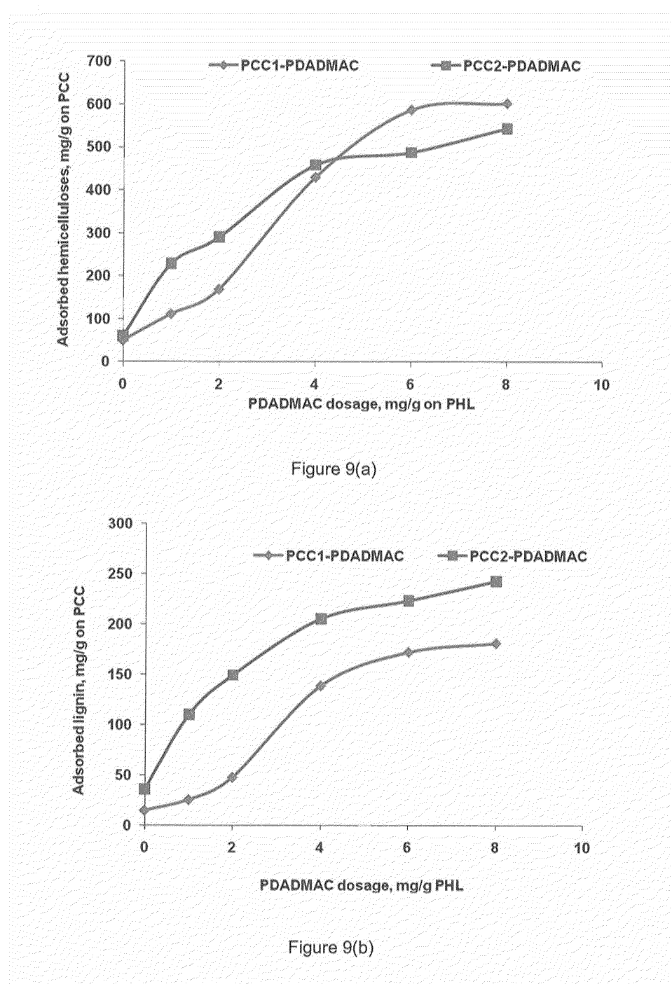

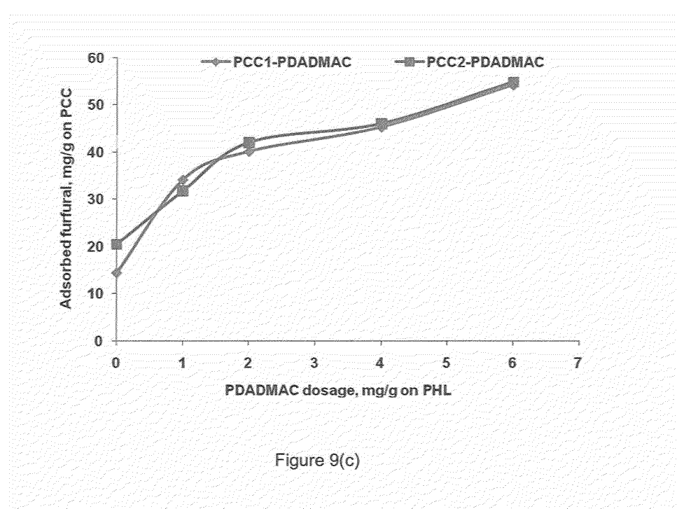

The results showed a marginal adsorption of acetic acid on precipitated calcium carbonate, regardless of the PDADMAC application. The adsorptions of lignocellulosic materials are presented in FIG. 9. As can be seen in FIG. 9(a), the adsorption of hemicelluloses increased from 100 mg/g to 500-600 mg/g on precipitated calcium carbonate by adding up to 8 mg/g PDADMAC on pre-hydrolysis liquor. The adsorption of lignin increased from 40 to 250 mg/g on PCC2, while it increased from 20 to 160 mg/g on PCC1. The adsorption of furfural was increased from 10-20 mg/g to 55 mg/g on precipitated calcium carbonate.

### Example 3

### Extraction of Lignocellulosic Materials of Pre-Hydrolysis Liquor Via Using Lime/Lime Mud

### Characterization

Pre-hydrolysis liquor was collected from the commercial plant that produces dissolving pulp based on the Kraft process (the same company described in Example 1). Its original pH was 4.2. Lime mud was also received from the same company. Calcium oxide used in Example 1 was used as a lime source in this experiment. PDADMAC that was used in Example 1 was applied in this example. The lignocelluloses content of pre-hydrolysis liquor was determined as described in Example 1.

### Removal Efficiency of Lime/Lime Mud

In this experiment, the pre-hydrolysis liquor was treated with various amounts of lime, lime mud and PDADMAC at 78° C. for 1 h. the pH of the experiment was adjusted to 11-11.2 by adding various dosages of lime/lime mud. The lignin and hemicelluloses contents of the pre-hydrolysis liquor after this treatment are listed in Table 1.

In one set of experiments, 1.3% (wt.) lime was added to the pre-hydrolysis liquor (No. 1), and the lignin and hemicelluloses removals were determined (Table 1). Evidently, the lignin and hemicelluloses removals were 25% and 20%, respectively.

In another set of experiments, lime and lime mud were applied together, but the controlling parameter of the system was the pH at 11. As can be seen, by treating the pre-hydrolysis liquor with 0.88% (wt. on pre-hydrolysis liquor) lime and 5% (wt. on pre-hydrolysis liquor) lime mud (No. 2), the lignin and hemicelluloses were removed by 33% and 7%, respectively.

By adding 0.05% (wt. on pre-hydrolysis liquor) PDADMAC to this system (No. 3), the lignin and hemicelluloses removals were improved to 28% and 39.4%, respectively. Thus, the addition of PDADMAC assisted the hemicelluloses removal, but insignificantly affected the lignin removal.

In another set of experiments (No. 4), the dosage of lime, lime mud and PDADMAC were 1.8, 5, and 0.05% (wt.) based on the pre-hydrolysis liquor. The results showed that the lignin and hemicelluloses were removed by 28% and 52%, respectively. Consequently, by increasing the dosage of lime, lime mud and PDADMAC, the lignin removal was insignificantly affected, but the hemicelluloses removal can be remarkably increased.

TABLE 1
Lignin and hemicelluloses contents of pre-hydrolysis liquor after treating
with lime/lime mud and PDADMAC at 78° C. for 1 h (pH 11)

| | Lime, | Lime | PDADMAC, | | |
|---|---|---|---|---|---|
| pre- | % (wt.) | mud, % | % (wt.) on | | |
| hydrolysis | on pre- | (wt.) on pre- | pre- | | Hemi- |
| liquor | hydrolysis | hydrolysis | hydrolysis | Lignin, | celluloses, |
| sample | liquor | liquor | liquor | g/l | g/l |
| Original | 0 | 0 | 0 | 9.2 | 50.4 |
| No. 1 | 1.3 | 0 | 0 | 6.9 | 40.0 |
| No. 2 | 0.88 | 5 | 0 | 6.1 | 46.8 |
| No. 3 | 0.88 | 5 | 0.05 | 6.6 | 30.5 |
| No. 4 | 1.8 | 5 | 0.05 | 6.6 | 24.2 |

### Example 4

### Step 1 of the Combined Process: Activated Carbon Adsorption

An industrially produced activated carbon was added to industrially produced pre-hydrolysis liquor, and the mixture was shaken at 150 rpm and room temperature for 5 h. Subsequently, the mixture was filtered using a Nylon 66 membrane with a pore size of 0.45 μm and diameter of 47 mm. Two weight ratios of activated carbon to pre-hydrolysis liquor, i.e., 1/30 and 1/40 were tested. Lignin concentration was measured by using a UV/Vis spectrometric method at a wavelength of 205 nm according to Tappi UM 250. The sugar concentration was measured by using an ion chromatography unit equipped with CarboPac™ PA1 column (Dionex-300, Dionex Corporation, USA) and a pulsed amperometric detector (PAD). It should be noted that C/Co refers to the ratio of lignocellulose concentration of treated pre-hydrolysis liquor to that of untreated pre-hydrolysis liquor.

The main components of pre-hydrolysis liquor are listed in Table 2[3]. As shown in Table 2 for the industrially produced pre-hydrolysis liquor used in this example, the concentration of sugars was the highest (around 50 g/l), followed by acetic acid and lignin.

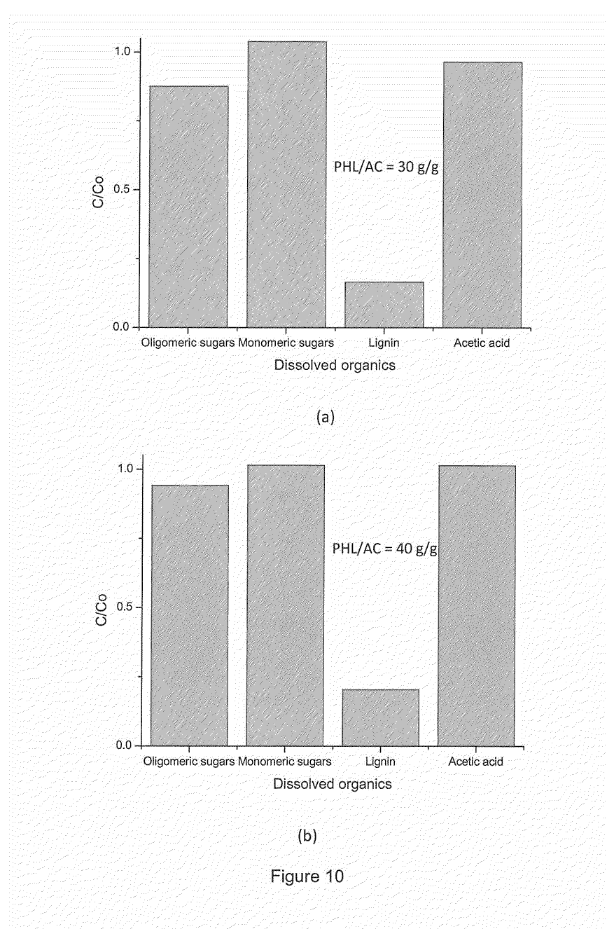

The effect of activated carbon treatment on the concentrations of oligomeric sugars, monomeric sugars, lignin, and acetic acid is shown in FIG. 10. It should be noted that C/Co refers to the ratio of lignocellulose concentration of treated pre-hydrolysis liquor to that of untreated pre-hydrolysis liquor. As seen from FIGS. 10(a) and 10(b), where the weight ratios of activated carbon to pre-hydrolysis liquor were 1/30 and 1/40, respectively, lignin was significantly removed while the removal of oligomeric sugars, monomeric sugars, and acetic acid was minimal.

For the dissolved organics in pre-hydrolysis liquor or other similar biomass pre-extract, lignin usually has a relatively high molecular weight. Therefore, by removing a substantial amount of lignin from the pre-hydrolysis liquor, the subsequent concentration of dissolved lower-molecular weight organics from the pre-hydrolysis liquor using membrane filtration technology (e.g., nanofiltration membrane) is facilitated.

TABLE 2
Compositions (g/l) of industrially produced pre-hydrolysis liquor [7]

| | | |
|---|---|---|
| Lignin | | 9.22 |
| Rhamnose | Monomeric | 0.44 |
| | Oligomeric | 0.28 |
| Arabinose | Monomeric | 0.85 |
| | Oligomeric | 0 |
| Galactose | Monomeric | 0.70 |
| | Oligomeric | 1.72 |
| Glucose | Monomeric | 1.16 |
| | Oligomeric | 6.61 |
| Xylose | Monomeric | 4.99 |
| | Oligomeric | 30.94 |
| Mannose | Monomeric | 0.26 |
| | Oligomeric | 2.38 |

| | |
|---|---|
| Total sugars | 50.33 |
| (both monomeric and oligomeric) | |
| Acetic acid | 10.11 |

### Example 5

### Step 1 of the Combined Process: Anion Exchange Resin Treatment

After activated carbon treatment (amount of treated pre-hydrolysis liquor to activated carbon of 30 g/g) as mentioned in Example 4, the pre-hydrolysis liquor was then treated using an weak base anion exchange resin (weight ratio of resin to pre-hydrolysis liquor: 1/10). After the resin was added to the concentrate, the mixture was shaken at 150 rpm and room temperature for 5 h. Subsequently, the mixture was filtered through a Nylon 66 membrane with a pore size of 0.45 μm and diameter of 47 mm.

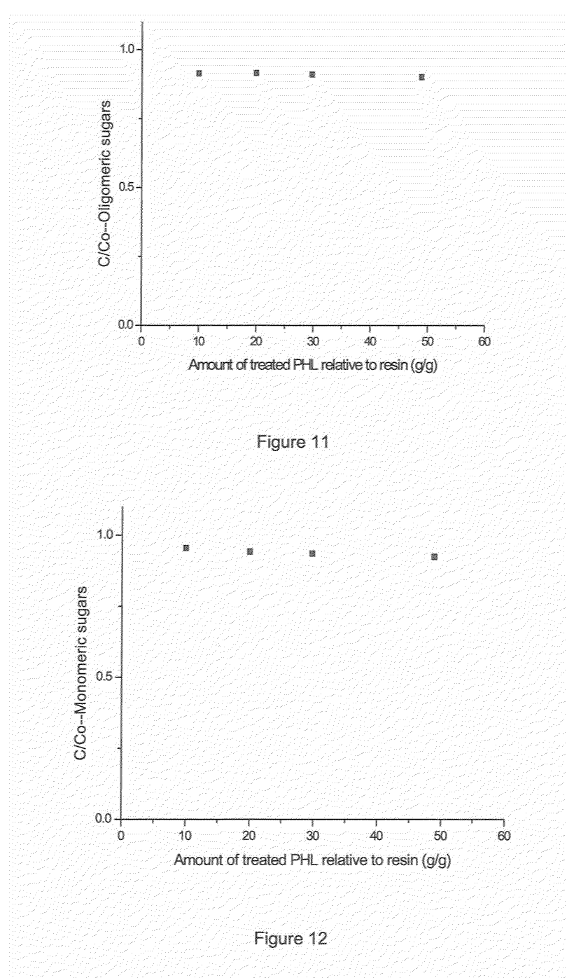

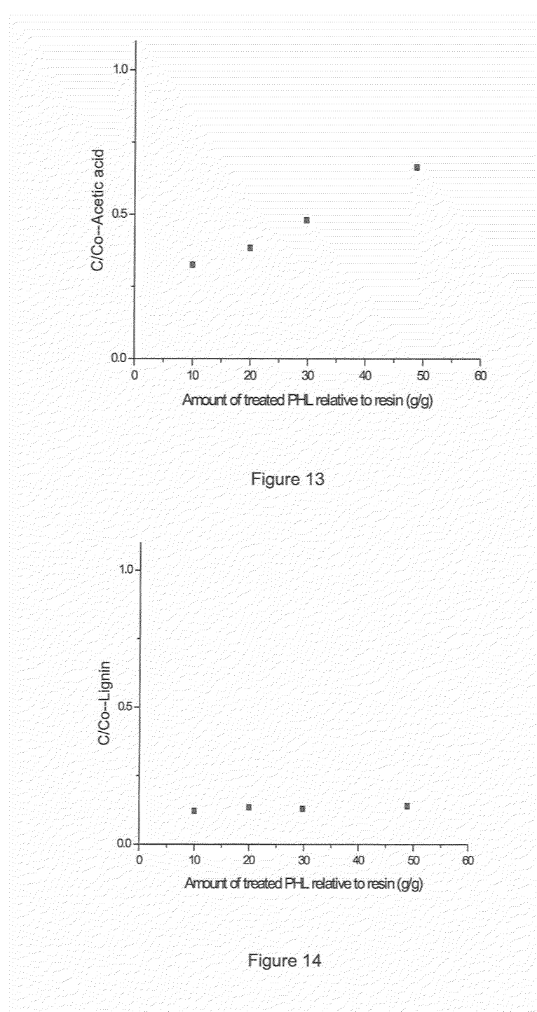

The effect of anion exchange resin treatment on the concentrations of oligomeric sugars, monomeric sugars, acetic acid, and lignin in pre-hydrolysis liquor is shown in FIGS. 11-14. As shown in the Figures, anion exchange resin treatment provides significant removal of acetic acid. For example, when the amount of treated pre-hydrolysis liquor relative to anion exchange resin was 10 g/g, the ratio of acetic acid concentration of treated pre-hydrolysis liquor to that of untreated pre-hydrolysis liquor was as low as about 0.3, as shown in FIG. 13. On the other hand, anion exchange resin treatment only had negligible effects on the concentrations of oligomeric sugars, monomeric sugars, and lignin, as shown in FIGS. 11, 12 and 14. Therefore, the acetic acid attached to anion exchange resin was in relatively pure form, which might be recovered and utilized by destroying the connection between acetic acid and resin. The recovery of acetic acid can also serve the purpose of resin regeneration/reactivation.

### Example 6

### Step 3 of the Combined Process: Membrane Concentration

After the sequential steps of activated carbon adsorption (amount of treated pre-hydrolysis liquor relative to activated carbon: 30 g/g) and anion exchange resin treatment (amount of treated pre-hydrolysis liquor relative to resin: 10 g/g) as mentioned in Examples 1 and 2, the double-treated pre-hydrolysis liquor was concentrated by using a nanofiltration membrane. The membrane filtration was conducted using a Sterlitech HP4750 stirred cell. The membrane was pre-wetted using deionized water and then loaded to the stirred cell. Then, 50 g deionized water was added to cell, and the system was gradually pressurized to 600 psi to allow deionized water to completely pass through the membrane under stirring. Subsequently, the passing of 50 g double-treated pre-hydrolysis liquor through the membrane was tested under the same condition. Similarly, the passing of 50 g untreated pre-hydrolysis liquor through the membrane was also tested.

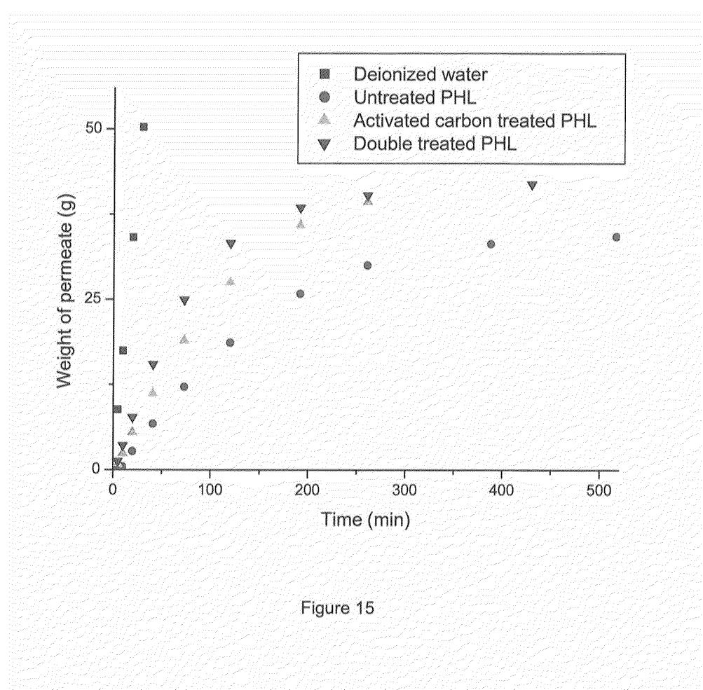

The nanofiltration membrane filterabilities of deionized water, untreated pre-hydrolysis liquor, activated carbon treated pre-hydrolysis liquor, and double-treated pre-hydrolysis liquor are shown in FIG. 15. For the three solutions, the filterability of deionized water was the best, while the filterability of untreated pre-hydrolysis liquor was the poorest. Interestingly, the filterabilities of activated carbon treated pre-hydrolysis liquor and double-treated pre-hydrolysis liquor were much better than that of untreated pre-hydrolysis liquor, predominately due to the removal of high-molecular-weight lignin from the pre-hydrolysis liquor as a result of activated carbon adsorption. The filterability of double-treated pre-hydrolysis liquor was slightly better than that of activated carbon treated pre-hydrolysis liquor, which could be due to the fact that the ion exchange resin treatment resulted in further removal of dissolved organics.

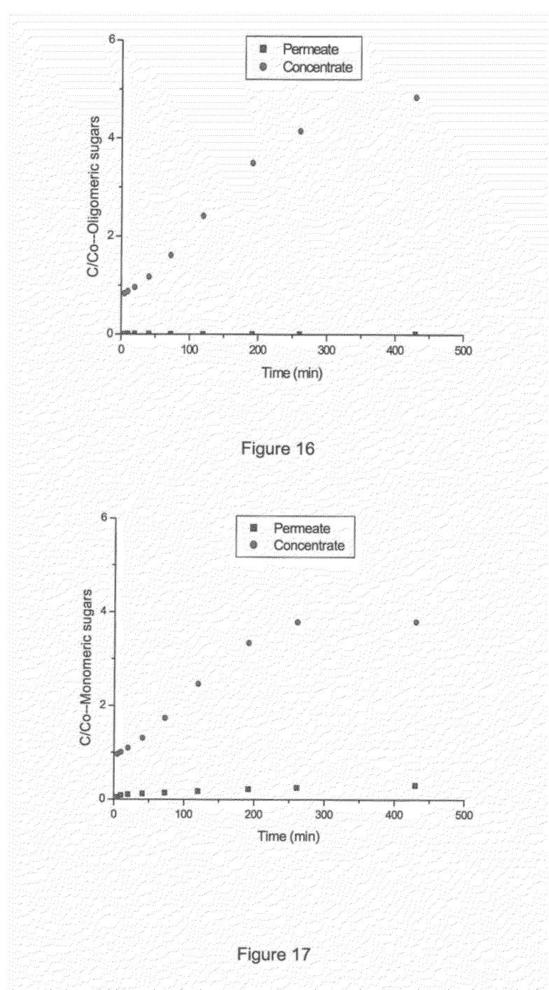

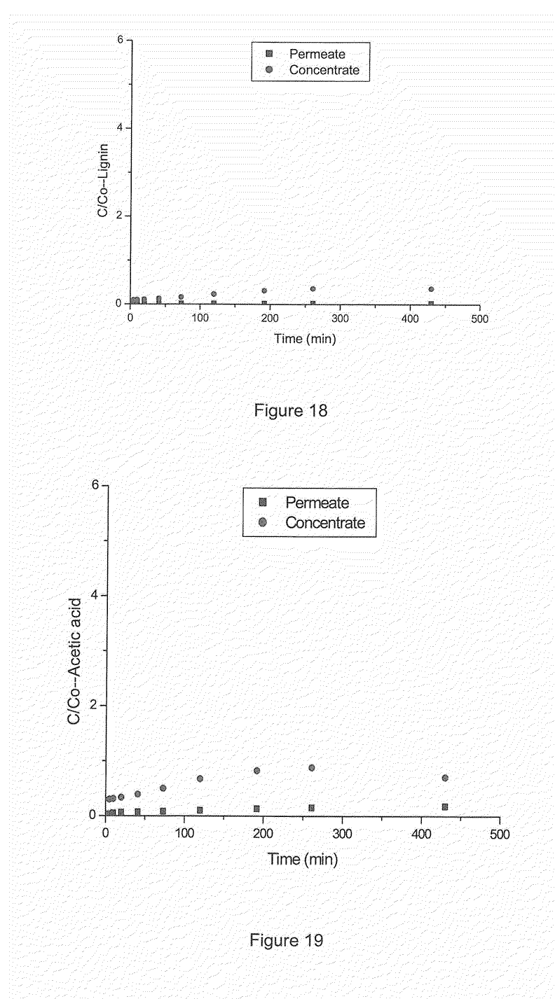

For the double-treated pre-hydrolysis liquor, during the nanofiltration process, the concentration variations of dissolved organics in the concentrate and permeate as a function of time are shown in FIGS. 16-19. The hemicellulose sugars, including oligomeric sugars and monomeric sugars, were significantly concentrated as a result of membrane filtration. After conducting the membrane filtration for 430 min, the concentration of total hemicellulose sugars in the concentrate was as high as 22.13%, whereas the concentrations of UV lignin and acetic acid in the final concentrate were 0.32% and 0.71%, respectively. On balance, the membrane filtration exhibited high efficiency in concentrating the dissolved organics in double-treated pre-hydrolysis liquor. This concentrated sugar stream can then be used to produce value-added products, such as furfural, xylitol, ethanol, and others.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

### REFERENCES

[1] Sixta, H. Handbook of Pulp. WILEY-VCH Verlag GmbH& Co. KGaA, Weinheim, Germany, 2006.
[2] Li, H., Saeed, A., Ni, Y., van Heiningen, A. R. P. Hemicellulose removal from hardwood chips in the pre-hydrolysis step of the kraft-based dissolving pulp production process. J. Wood Chem. Technol. 30(1), 48-60 (2010).
[3] Shen, J.; Fatehi, P.; Soleimani, P.; Ni, Y. Recovery of lignocelluloses from pre-hydrolysis liquor in the lime kiln of kraft-based dissolving pulp production process by adsorption to lime mud. Bioresour. Technol. 102(21), 10035-10039 (2011)
[4] Mussatto S I, Fernandes M, Roberto I C. Lignin recovery from brewer's spent grain black liquor. Carbohydr. Polym. 2007; 70(2):218-223.
[5] Li H. Saeed A, Jahan S M, Ni Y, van Heiningen A. Hemicellulose removal from hardwood chips in the pre-hydrolysis step of the Kraft-based dissolving pulp production process. J. Wood Chem. Technol. 2010; 30(1):48-60.
[6] Ghosh P K, Hexavalent chromium [Cr(VI)] removal by acid modified waste activated carbons. J. Hazard. Materials 2009; 171(1-3):116-122.
[7] Saeed A, Jahan M S, Li H, Liu Z, Ni Y, van Heiningen A. Mass balances of components dissolved in the pre-hydrolysis liquor of Kraft-based dissolving pulp production process from Canadian hardwoods, Biomass Bioen. doi:10.1016/j.biombioe.2010.08.039
[8] Ni Y, Kang G J, van Heiningen A. Are hydroxyl radicals responsible for degradation of carbohydrates during ozone bleaching of chemical pulp?. J. Pulp Paper Sci. 1996; 22(2):53-57.
[9] Ni Y, Kang G. Formation of peracetic acid during peroxide bleaching of mechanical pulps. Appita 2007; 60(1):70-73
[10] van de Ven T G M. Kinetic aspects of polymer and polyelectrolyte adsorption on surfaces. Adv. Colloid Interface Sci. 1994; 48:121-140.
[11] Hsieh J S, Yoo S. Adsorption and Recovery of Nonionic Polymers by Neutralization of Cellulose Fiber Surface Charge via Cationic Polyamide-Epichlorohydrin Resins. J. Appl. Polym. Sci. 2010; 117(3):1476-1485.
[12] Nurmi M, Eklund D. Effect of cationic polyacrylamide on colloidal wood resin. Paper Timber 2000; 82(5):331-334.

Therefore what is claimed is:

1. A method of treating a solution containing lignocellulosic material, the method comprising the steps of:
providing a quantity of activated carbon;
producing modified activated carbon by contacting the activated carbon with one of an oxidizing agent and a cationic polymer;
mixing the modified activated carbon with the solution for a time duration suitable for adsorbing lignocellulosic material onto the modified activated carbon; and
separating the modified activated carbon having the lignocellulosic material adsorbed thereon from the solution.

2. The method according to claim 1 wherein the oxidizing agent is selected from the group consisting of hydrogen peroxide, sulfuric acid, peroxy acid, and nitric acid.

3. The method according to claim 1 wherein the oxidizing agent is hydrogen peroxide, wherein the step of contacting the activated carbon with the oxidizing agent includes mixing the activated carbon in a hydrogen peroxide solution having a hydrogen peroxide concentration between approximately 0.1 to 10 g/l, for a time duration of approximately 5 to 90 minutes, and at a temperature between approximately 25 to 70 degrees centigrade, wherein a weight percentage of activated carbon to hydrogen peroxide solution is between approximately 1 to 20%.

4. The method according to claim 1 wherein the cationic polymer is selected from the group consisting of PDADMAC and chitosan.

5. The method according to claim 1 wherein the cationic polymer is selected from the group consisting of poly acrylamide, poly aluminum chloride, amine-based cationic polymers, and combinations thereof.

6. The method according to claim 1 wherein the activated carbon is contacted with approximately 2 to 2.5 milligrams of cationic polymer per gram of activated carbon.

7. The method according to claim 1 wherein the solution is a pulp production waste liquor.

8. The method according to claim 7 wherein the pulp production waste liquor is a prehydrolysis liquor.

9. The method according to claim 1 further comprising the step of desorbing the lignocellulosic material from the modified activated carbon after the step of separating the modified activated carbon from the solution.

10. The method according to claim 9 further comprising the step of recovering the modified activated carbon after the step of desorbing the lignocellulosic material.

11. The method according to claim 1 further comprising the step of burning, in a gasifier, the modified activated carbon having the lignocellulosic material adsorbed thereon.

12. The method according to claim 11 wherein further comprising the step extracting energy released from the step of burning the modified activated carbon having the lignocellulosic material adsorbed thereon.

13. The method according to claim 11 further comprising the step of recovering the modified activated carbon after the step of burning the modified activated carbon.

14. The method according to claim 1 wherein approximately 1 to 100 grams of modified activated carbon are provided per liter of the solution.

15. The method according to claim 1 wherein prior to the step of mixing the modified activated carbon with the solution, the following steps are performed:
acidifying the solution and obtaining precipitated lignin;
extracting the precipitated lignin; and
neutralizing the solution.

16. The method according to claim 1 further comprising the step of modifying a pH of the solution to a value within a range of approximately 5 to 10 prior to mixing the modified activated carbon with the solution.

17. A method of extracting lignocellulosic organic material from a lignocellulosic solution, the method comprising the steps of:
providing a quantity of calcium carbonate;
forming a mixture including the lignocellulosic solution and calcium carbonate;
providing a quantity of a cationic polymer, the cationic polymer being selected for inducing flocculation of the lignocellulosic organic material;
adding the cationic polymer to the mixture and mixing for a time duration suitable for adsorbing lignocellulosic organic material onto the calcium carbonate; and
separating the calcium carbonate from the mixture, thereby obtaining calcium carbonate having the lignocellulosic organic material adsorbed thereon.

18. The method according to claim 17 wherein the lignocellulosic solution is a pulp production waste liquor.

19. The method according to claim 18 wherein the pulp production waste liquor is a prehydrolysis liquor.

20. The method according to claim 17 further comprising the step of desorbing the lignocellulosic organic material from the calcium carbonate after the step of separating the calcium carbonate from the mixture.

21. The method according to claim 17 wherein the quantity of the calcium carbonate is approximately 0.5 to 100 grams of calcium carbonate per liter of the lignocellulosic solution.

22. The method according to claim 17 wherein the cationic polymer is selected from the group consisting of PDADMAC and chitosan.

23. The method according to claim 17 wherein the cationic polymer is selected from the group consisting of poly acrylamide, poly aluminum chloride, amine-based cationic polymers, and combinations thereof.

24. The method according to claim 17 wherein the quantity of the cationic polymer is approximately 0.1 to 10 milligrams of cationic polymer per liter of the lignocellulosic solution.

25. A method of extracting lignocellulosic organic material from a lignocellulosic solution, the method comprising the steps of:
providing a quantity of lime;
forming a mixture including the lignocellulosic solution and the lime;
providing a quantity of a cationic polymer, the cationic polymer being selected for inducing flocculation of the lignocellulosic organic material;
adding the cationic polymer to the mixture and mixing for a time duration suitable for adsorbing lignocellulosic organic material onto the lime; and
separating the lime from the mixture, thereby obtaining calcium carbonate having the lignocellulosic organic material adsorbed thereon.

26. The method according to claim 25 wherein the lignocellulosic solution is a pulp production waste liquor.

27. The method according to claim 26 wherein the pulp production waste liquor is a prehydrolysis liquor.

28. The method according to claim 25 further comprising the step of adding lime mud to the mixture such that the lignocellulosic organic material is also adsorbed onto the lime mud, and subsequently separating the lime mud from the mixture.

29. The method according to claim 25 further comprising the step of desorbing the lignocellulosic organic material from the lime after the step of separating the lime from the mixture.

30. The method according to claim 29 where the step of desorbing the lignocellulosic organic material is performed using a solvent extraction unit.

31. The method according to claim 29 further comprising the step of recovering the lime after the step of desorbing the lignocellulosic organic material.

32. The method according to claim 25 further comprising the step of burning separated lime for extraction of energy.

33. The method according to claim 32 wherein the step of burning separated lime includes recovering calcium oxide.

34. The method according to claim 25 wherein the quantity of the lime is approximately 0.5 to 100 grams of lime per liter of the lignocellulosic solution.

35. The method according to claim 25 wherein the quantity of the cationic polymer is approximately 0.1 to 10 milligrams of cationic polymer per liter of the lignocellulosic solution.

36. The method according to claim 25 wherein the quantity of the lime mud is approximately 0 to 5 percent by weight.

37. A method of treating a lignocellulosic solution, the lignocellulosic solution containing lignin, acetic acid, and hemicellulose sugars, the method comprising the steps of:
providing a quantity of adsorbent configured to adsorb the lignin;
mixing adsorbent with the lignocellulosic solution for a time duration suitable for adsorbing a substantial portion of the lignin onto the adsorbent;
separating the adsorbent from the lignocellulosic solution;
providing a quantity of an ion exchange resin, wherein the ion exchange resin is configured to adsorb acetic acid;
contacting the ion exchange resin with the lignocellulosic solution and extracting acetic acid onto the ion exchange resin;
separating the ion exchange resin from the lignocellulosic solution;
providing a filter configured to capture hemicellulose sugars; and
filtering the lignocellulosic solution with the filter, thereby concentrating the hemicellulose sugars;
wherein the step of mixing the adsorbent with the lignocellulosic solution is performed prior to the steps of contacting the ion exchange resin with the lignocellulosic solution and filtering the lignocellulosic solution with the filter, such that the lignin is removed prior to the removal of acetic acid and hemicellulose sugars.

38. The method according to claim 37 wherein the step of filtering the lignocellulosic solution with the filter is performed prior to the step of contacting the ion exchange resin with the lignocellulosic solution and extracting acetic acid onto the ion exchange resin.

39. The method according to claim 37 wherein the filter is a membrane filter.

40. The method according to claim 37 wherein the filter selected from the group consisting of an ultrafiltration filter, a nanofiltration filter, and a reverse osmosis membrane filter.

41. The method according to claim 37 wherein the lignocellulosic solution is a pre-hydrolysis liquor from a kraft-based dissolving pulp production process.

42. The method according to claim 41 wherein a solids content of the pre-hydrolysis liquor is approximately 4-12% by weight.

43. The method according to claim 41 wherein a pH of the pre-hydrolysis liquor is within the range of approximately 3-5.

44. The method according to claim 41 wherein an amount of the acetic acid relative to a total amount of dissolved organics within the pre-hydrolysis liquor is approximately 15-25% by weight.

45. The method according to claim 41 wherein an amount of the hemicellulose sugars relative to a total amount of dissolved organics within the pre-hydrolysis liquor is approximately 25-45% by weight.

46. The method according to claim 37 wherein the adsorbent is activated carbon.

47. The method according to claim 46 wherein the activated carbon is modified prior to the step of mixing the adsorbent with the lignocellulosic solution by contacting the activated carbon with one of an oxidizing agent and a cationic polymer.

48. The method according to claim 37 further comprising the step of desorbing the lignin from the adsorbent after separating the adsorbent from the lignocellulosic solution.

49. The method according to claim 37 further comprising the step of recovering the acetic acid from the ion exchange resin after separating the ion exchange resin from the lignocellulosic solution.

50. The method according to claim 37 further comprising the step of recovering the hemicellulose sugars from the filter after filtering the lignocellulosic solution.
